(19) United States
(12) Patent Application Publication
(10) Pub. No.: US 2024/0229154 A9
(43) Pub. Date: Jul. 11, 2024

(54) METHOD FOR PROGNOSIS AND TREATING A PATIENT SUFFERING FROM CANCER

(71) Applicants: INSTITUT NATIONAL DE LA SANTÉ ET DE LA RECHERCHE MÉDICALE, Paris (FR); SORBONNE UNIVERSITÉ, Paris (FR); UNIVERSITE PARIS CITE, Paris (FR)

(72) Inventors: Lubka ROUMENINA, Paris (FR); Marie DAUGAN, Paris (FR); Margot REVEL, Paris (FR); Isabelle CREMER, Paris (FR); Catherine SAUTES-FRIDMAN, Paris (FR); Wolf Herman FRIDMAN, Paris (FR)

(21) Appl. No.: 18/546,028

(22) PCT Filed: Feb. 11, 2022
(86) PCT No.: PCT/EP2022/053325

§ 371 Date: Aug. 10, 2023

(30) Foreign Application Priority Data
Feb. 12, 2021 (EP) 21305187.3

(51) Int. Cl. C12Q 1/6886; C12Q 1/6874; G01N 33/574

(52) U.S. Cl. CPC C12Q 1/6886; C12Q 1/6874; G01N 33/57496; C12Q 2600/106; C12Q 2600/118; C12Q 2600/158; G01N 2333/4716

(57) ABSTRACT

Complement components form a plasma innate immune cascade but could also serve as multitasking proteins, as they have functions beyond this system. Here, we show that complement FH is locally expressed by multiple types of human tumors. We provide a paradigm shift for the impact of FH on cancer progression, showing a previously unrecognized, intracellular function of FH outside the complement cascade, while the canonical, complement-regulatory function had no effect. Int-FH served as a driver of the proliferation and migration of ccRCC and lung ADK cells but not of normal cells or lung SCC cells. The presence of int-FH staining in tumor cells indicated poor prognosis for ccRCC and lung ADK. Thus, the invention relates to a method for predicting the survival time of a patient suffering from a cancer, comprising i) determining in a sample obtained from the patient the expression level of int-FH ii) comparing the expression level determined at step i) with its predetermined reference value and iii) providing a prognosis when the expression level determined at step i) is modulated compared to its predetermined reference value.

### FIELD OF THE INVENTION

The present invention is in the field of oncology. In particular, the invention relates to a method for predicting the survival time of a patient suffering from a cancer.

### BACKGROUND OF THE INVENTION

The complement cascade is a part of innate immunity¹, it kills pathogens and helps to maintain host homeostasis². Nowadays there is an effervesces of complement therapeutics entering clinical trials³. Therefore, it is critical to know whether and how complement proteins affect diseases in order to propose adapted therapeutic solutions. Tumors are a complement-rich environment⁴ in which numerous cell types, such as immune cells⁵, endothelial cells^{6,7}, fibroblasts⁸ or the malignant cells themselves^{4,7} contribute to the local production of complement components. To turn complement activation in their favor, tumor cells develop mechanisms to escape the canonical functions of the complement cascade⁴.

FH is the master regulator of the central enzyme of the complement alternative pathway (AP): C3 convertase. The canonical function of FH is to block C3 convertase formation, enhance its dissociation and exert cofactor activity for FI, an enzyme cleaving C3b to inactive fragment iC3b¹. The presence of FH in a tumor can come from circulation or from in situ production by tumor cells. Although studies in patients are scarce, animal models and in vitro studies revealed that within the tumor microenvironment (TME), FH could have context-dependent action. Due to the overexpression of complement regulators, such as FH, opsonization with C3b and lytic membrane attack complex (MAC) formation are hampered on tumor cells⁹⁻¹¹, while chronic inflammation via the generation of anaphylatoxins continues¹²⁻¹⁶ In line with these observations, injection of a blocking FH antibody also reduced lung cancer growth in mice¹⁷. In other conditions, FH had an antitumoral effect by regulating the generation of anaphylatoxins, which were shown to promote an immunosuppressive environment, as in a mouse model of sarcoma¹⁶. Moreover, spontaneous hepatic tumor formation was found in aged mice with FH deficiency¹⁸.

It is now clear that cancer cells produce complement proteins, which are not all secreted but partially remain within the cell¹⁹. The role of these intracellular complement proteins in cancer is unknown, but in T cells or in pancreatic islets, C3 and/or C5 have intracellular action outside of the complement cascade, termed noncanonical functions²⁰⁻²². Intracellular staining of FH (int-FH) has been reported for ovarian cancer cells²³, glioma cells^{24,25} cutaneous squamous cell carcinoma (cSCC) cells²⁶, breast cancer²⁷ and lung cancer cells¹⁰, but the function and relevance of this FH for tumor progression were poorly understood. However, recent studies have shown that FH can have an intrinsic role independent of the complement cascade²⁸. In liver cancer, FH is required for the maintenance of stemness²⁹. It promoted immunosuppressive phenotype of macrophages in the context of breast cancer²⁷. Importantly, in cSCC, silencing FH production in tumor cells decreased proliferation and migration and altered cell signaling²⁶, but it is unclear whether these biological functions are related to extracellular or intracellular effects. Taking into account these diverse and context-dependent and often contradictory modes of action of FH described in vitro and in mice, it was critical to provide assessment of its role in human cancer in order to understand its biological functions and shed light on its usefulness as a prognostic biomarker or therapeutic target.

### SUMMARY OF THE INVENTION

Interestingly, the inventors showed that FH was present in two distinct compartments: extracellular (ext-FH) and intracellular (int-FH), and that int-FH exerts protumoral action through a noncanonical mechanism, acting on proliferation, cell cycle, morphology and migration, according to cell-types specific effects. While ext-FH performed its canonical functions of complement regulation with no impact on the tumor cell phenotype or patient survival, it was demonstrated that int-FH is associated with poor prognosis in patient cohorts. This discovery highlights an unexpected role of int-FH in tumor progression and permits to develop its use as prognostic biomarker and therapeutic target. Thus, the present invention relates to a method for predicting the survival time of a patient suffering from a cancer, comprising i) determining in a sample obtained from the patient the expression level of int-FH ii) comparing the expression level determined at step i) with its predetermined reference value and iii) providing a prognosis when the expression level determined at step i) is modulated compared to its predetermined reference value.

### DETAILED DESCRIPTION OF THE INVENTION

The inventors showed that int-FH is overexpressed by tumor cells in a subset of patients with lung adenocarcinoma (ADK) and clear cell renal cell carcinoma (ccRCC) and is associated with poor prognosis.

Prognosis Method of the Invention

A first aspect of the present invention relates to a method for predicting the survival time of a patient suffering from a cancer, comprising i) determining in a sample obtained from the patient the expression level of int-FH ii) comparing the expression level determined at step i) with its predetermined reference value and iii) providing a prognosis when the expression level determined at step i) is modulated compared to its predetermined reference value.

In some embodiments, the present invention relates to a method for predicting the survival time of a patient suffering from a cancer, comprising i) determining in a sample obtained from the patient the expression level of int-FH ii) comparing the expression level determined at step i) with its predetermined reference value and iii) providing a bad prognosis when the expression level determined at step i) is higher than its predetermined reference value, or providing a good prognosis when the expression level determined at step i) is lower than its predetermined reference value.

In a particular embodiment, the cancer is a lung adenocarcinoma or a renal carcinoma. In a more particular embodiment, the renal carcinoma is a clear cell renal cell carcinoma.

Thus, in some embodiments, the present invention relates to a method for predicting the survival time of a patient suffering from a lung adenocarcinoma or a clear cell renal cell carcinoma, comprising i) determining in a sample obtained from the patient the expression level of int-FH ii) comparing the expression level determined at step i) with its predetermined reference value and iii) providing a bad prognosis when the expression level determined at step i) is higher than its predetermined reference value, or providing a good prognosis when the expression level determined at step i) is lower than its predetermined reference value.

In some embodiments, the present invention relates to a method for predicting the survival time of a patient suffering from a cancer, comprising i) determining in a sample obtained from the patient the expression level of int-FH ii) comparing the expression level determined at step i) with its predetermined reference value and iii) providing a good prognosis when the expression level determined at step i) is higher than its predetermined reference value, or providing a bad prognosis when the expression level determined at step i) is lower than its predetermined reference value.

In some embodiments, the cancer is a liver cancer.

Thus, in some embodiments, the present invention relates to a method for predicting the survival time of a patient suffering from a liver cancer, comprising i) determining in a sample obtained from the patient the expression level of int-FH ii) comparing the expression level determined at step i) with its predetermined reference value and iii) providing a good prognosis when the expression level determined at step i) is higher than its predetermined reference value, or providing a bad prognosis when the expression level determined at step i) is lower than its predetermined reference value.

As used herein, the term “patient” or “subject” refers to any mammals, such as a rodent, a feline, a canine or a primate. In some embodiments, the patient is a human afflicted with a cancer. In some embodiments, the patient is a human afflicted with a lung, renal or liver cancer. In some embodiments, the patient is a human afflicted with a lung or renal cancer.

As used herein, the term “cancer” has its general meaning in the art and includes, but is not limited to, solid tumors and blood borne tumors. The term cancer includes diseases of the skin, tissues, organs, bone, cartilage, blood and vessels. The term “cancer” further encompasses both primary and metastatic cancers. Examples of cancers include, but are not limited to, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

As used herein, the term “survival time” denotes the percentage of people in a study or treatment group who are still alive for a certain period of time after they were diagnosed with or started treatment for a disease, such as lung adenocarcinoma or a renal carcinoma. The term “survival time” can regroup the terms overall survival (OS), progression-free survival (PFS) and/or the disease-free survival (DFS). Those of skill in the art will recognize that OS survival time is generally based on and expressed as the percentage of people who survive a certain type of cancer for a specific amount of time. Cancer statistics often use an overall five-year survival rate. In general, OS rates do not specify whether cancer survivors are still undergoing treatment at five years or if they have become cancer-free (achieved remission). DSF gives more specific information and is the number of people with a particular cancer who achieve remission. Also, progression-free survival (PFS) rates (the number of people who still have cancer, but their disease does not progress) include people who may have had some success with treatment, but the cancer has not disappeared completely.

As used herein, the term “int-FH” refers to intracellular staining of FH, while the term “ext-FH” refers to extracellular staining of FH. More particularly, the term “int-FH” refers to intracellular staining of FH protein and “ext-FH” refers to extracellular staining of FH protein. Int-FH is encoded by CFH gene and performed noncanonical (e.g. intracellular action outside of the complement cascade²⁰⁻²²) functions of complement regulation, contrarily to ext-FH, which exerts canonical functions. Int-FH is reported for ovarian cancer cells²¹, glioma cells^{24,25}, cutaneous squamous cell carcinoma (cSCC) cells²⁶, breast cancer cells²⁷ and lung cancer cells¹⁰.

As used herein, the term “sample” refers to any substance of biological origins. As example, such sample can be a tissue sample or a body-fluid sample, more particularly a tumor sample, a cancer biopsy, a cancer surgical specimen, blood, peripheral-blood, serum or plasma.

In some embodiments, the sample is a tissue sample. As used herein, the term “tissue”, when used in reference to a part of a body or of an organ, generally refers to an aggregation or collection of morphologically similar cells and associated accessory and support cells and intercellular matter, including extracellular matrix material, vascular supply, and fluids, acting together to perform specific functions in the body. There are generally four basic types of tissue in animals and humans including muscle, nerve, epithelial, and connective tissues. In some embodiments, the tissue sample is a tumor sample containing tumor cells. In some embodiments, the tissue sample is a tumor sample containing lung adenocarcinoma, renal carcinoma cells or liver cancer cells. In some embodiments, the tissue sample is a tumor sample containing lung adenocarcinoma or renal carcinoma cells. In some embodiments, the renal carcinoma cells are clear cell renal cells. In some embodiments, the sample contains liposomes from cancer cells.

Measuring the expression level of int-FH can be performed by a variety of techniques well known in the art.

Distinction of the intracellular staining from membrane deposits has to be made by observation of the cells. In particular cases one cell can be positive both for intracellular protein staining (Int-FH positive) and also positive at the membrane (Ext-FH). The presence of ext-FH or not does not impact the evaluation of the Int-FH.

Thus, in some embodiments, the level of int-FH is determined by Immunohistochemistry (IHC). In some embodiments, the level of int-FH is determined by Immunohistochemistry (IHC) when the sample is a tumor sample. For example, the quantification of the level of int-FH is performed by contacting a tissue sample with binding partners (e.g. antibodies) specific for int-FH. Immunohistochemistry typically includes the following steps i) fixing the tissue sample with formalin, ii) embedding said tissue sample in paraffin, iii) cutting said tissue sample into sections for staining, iv) incubating said sections with the binding partner specific for the marker, v) rinsing said sections, vi) incubating said section with a secondary antibody typically biotinylated and vii) revealing the antigen-antibody complex typically with avidin-biotin-peroxidase complex. Accordingly, the tissue sample is firstly incubated with the binding partners, such as antibodies. In some embodiments, a further step is composed consisting in permeabilizing the tissue section. After washing, the labeled antibodies that are bound to marker of interest are revealed by the appropriate technique, depending of the kind of label is borne by the labeled antibody, e.g. radioactive, fluorescent or enzyme label. Multiple labelling can be performed simultaneously. Alternatively, the method of the present invention may use a secondary antibody coupled to an amplification system (to intensify staining signal) and enzymatic molecules. Such coupled secondary antibodies are commercially available, e.g. from Dako, EnVision system. Counterstaining may be used, e.g. Hematoxylin & Eosin, DAPI, Hoechst. Other staining methods may be accomplished using any suitable method or system as would be apparent to one of skill in the art, including automated, semi-automated or manual systems. For example, one or more labels can be attached to the antibody, thereby permitting detection of the marker. Exemplary labels include radioactive isotopes, fluorophores, ligands, chemiluminescent agents, enzymes, and combinations thereof. In some embodiments, the label is a quantum dot. Non-limiting examples of labels that can be conjugated to primary and/or secondary affinity ligands include fluorescent dyes or metals (e.g. fluorescein, rhodamine, phycoerythrin, fluorescamine), chromophoric dyes (e.g. rhodopsin), chemiluminescent compounds (e.g. luminal, imidazole) and bioluminescent proteins (e.g. luciferin, luciferase), haptens (e.g. biotin). A variety of other useful fluorescers and chromophores are described in Stryer L (1968) Science 162:526-533 and Brand L and Gohlke J R (1972) Annu. Rev. Biochem. 41:843-868. Affinity ligands can also be labeled with enzymes (e.g. horseradish peroxidase, alkaline phosphatase, beta-lactamase), radioisotopes (e.g. ³H, ¹⁴C, ³²P, ³⁵S or ¹²⁵I) and particles (e.g. gold). The different types of labels can be conjugated to an affinity ligand using various chemistries, e.g. the amine reaction or the thiol reaction. However, other reactive groups than amines and thiols can be used, e.g. aldehydes, carboxylic acids and glutamine. Various enzymatic staining methods are known in the art for detecting a protein of interest. For example, enzymatic interactions can be visualized using different enzymes such as peroxidase, alkaline phosphatase, or different chromogens such as DAB, AEC or Fast Red. In other examples, the antibody can be conjugated to peptides or proteins that can be detected via a labeled binding partner or antibody. In an indirect IHC assay, a secondary antibody or second binding partner is necessary to detect the binding of the first binding partner, as it is not labeled. The resulting stained specimens are each imaged using a system for viewing the detectable signal and acquiring an image, such as a digital image of the staining. Methods for image acquisition are well known to one of skill in the art. For example, once the sample has been stained, any optical or non-optical imaging device can be used to detect the stain or biomarker label, such as, for example, upright or inverted optical microscopes, scanning confocal microscopes, cameras, scanning or tunneling electron microscopes, canning probe microscopes and imaging infrared detectors. In some examples, the image can be captured digitally. The obtained images can then be used for quantitatively or semi-quantitatively determining the amount of the marker in the sample, or the absolute number of cells positive for the maker of interest, or the surface of cells positive for the maker of interest. Various automated sample processing, scanning and analysis systems suitable for use with IHC are available in the art. Such systems can include automated staining and microscopic scanning, computerized image analysis, serial section comparison (to control for variation in the orientation and size of a sample), digital report generation, and archiving and tracking of samples (such as slides on which tissue sections are placed). Cellular imaging systems are commercially available that combine conventional light microscopes with digital image processing systems to perform quantitative analysis on cells and tissues, including immunostained samples. See, e.g., the CAS-200 system (Becton, Dickinson & Co.). In particular, detection can be made manually or by image processing techniques involving computer processors and software. Using such software, for example, the images can be configured, calibrated, standardized and/or validated based on factors including, for example, stain quality or stain intensity, using procedures known to one of skill in the art (see e.g., published U.S. Patent Publication No. US20100136549). The image can be quantitatively or semi-quantitatively analyzed and scored based on staining intensity of the sample. Quantitative or semi-quantitative histochemistry refers to method of scanning and scoring samples that have undergone histochemistry, to identify and quantitate the presence of the specified marker. Quantitative or semi-quantitative methods can employ imaging software to detect staining densities or amount of staining or methods of detecting staining by the human eye, where a trained operator ranks results numerically. A ratio of strong positive stain (such as brown stain) to the sum of total stained area can be calculated and scored. The amount of the detected marker is quantified and given as a percentage of positive pixels and/or a score. For example, the amount can be quantified as a percentage of positive pixels. In some examples, the amount is quantified as the percentage of area stained, e.g., the percentage of positive pixels. For example, a sample can have at least or about at least or about 0, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 40%, 45%, 50%, 55%, 60%, 65%, 70%, 75%, 80%, 85%, 90%, 95% or more positive pixels as compared to the total staining area. In a preferred embodiment, the amount can be quantified as an absolute number of cells positive for the maker of interest. In some embodiments, a score is given to the sample that is a numerical representation of the intensity or amount of the histochemical staining of the sample, and represents the amount of target marker present in the sample. Optical density or percentage area values can be given a scaled score, for example on an integer scale. Thus, in some embodiments, the method of the present invention comprises the steps consisting in i) providing one or more immunostained slices of tissue section obtained by an automated slide-staining system by using a binding partner capable of selectively interacting with int-FH (e.g. an antibody as above described), ii) proceeding to digitalisation of the slides of step i).by high resolution scan capture, iii) detecting the slice of tissue section on the digital picture iv) providing a size reference grid with uniformly distributed units having a same surface, said grid being adapted to the size of the tissue section to be analyzed, and v) detecting, quantifying and measuring intensity or the absolute number of stained cells in each unit whereby the number or the density of cells stained of each unit is assessed. In some embodiments, the level of int-FH is determined by Immunofluorescence (IF). Immunofluorescence is an immunostaining technique, which uses antibodies coupled to fluorochromes. Immunofluorescence can reveal a specific protein directly in the cell, by fluorescence emission. It therefore makes it possible to determine the presence or absence of a protein, but also its location in the cell or the tissue analysed.

In a particular embodiment, the detection of the level of int-FH can be performed by flow cytometry. When this method is used, the method consists of determining the amount of int-FH expressed on tumor cells. According to the invention and the flow cytometry method, when the florescence intensity is high or bright, the level of int-FH express on tumor cells is high and thus the expression level of int-FH is high and when the florescence intensity is low or dull, the level of int-FH express on tumor cells is low and thus the expression level of int-FH is low. As example, flow cytometry can be used in fresh tumor samples, after permeabilization and comparison of the signal of non-permeabilized versus permeabilized tumor cells. The signal permeabilized—non-permeabilized give a value which indicate if the cell is intracellularly positive or not.

As intracellular biomarker, int-FH may be separated from ext-FH to measure its expression level according to the present prognosis method. As demonstrated in the present disclosure, int-FH is mainly present in organelles, in particular liposomes, and many methods are available to the man skilled in the art to isolate such molecule, as using a fractionation kit (Abcam, ab109719) providing enriched fractions of subcellular components.

For measuring the expression level of FH, techniques like ELISA (see below) allowing to measure the level of the soluble proteins are suitable.

In the present application, the “level of protein” or the “protein level expression” or the “protein concentration” means the quantity or concentration of said protein. In another embodiment, the “level of protein” means the level of FH proteins fragments. In still another embodiment, the “level of protein” means the quantitative measurement of FH proteins expression relative to a negative control. Typically, protein concentration may be measured for example by capillary electrophoresis-mass spectroscopy technique (CE-MS) or ELISA performed on the sample. Such methods comprise contacting a sample with a binding partner capable of selectively interacting with proteins present in the sample. The binding partner is generally an antibody that may be polyclonal or monoclonal, preferably monoclonal. The presence of the protein can be detected using standard electrophoretic and immunodiagnostic techniques, including immunoassays such as competition, direct reaction, or sandwich type assays. Such assays include, but are not limited to, Western blots; agglutination tests; enzyme-labeled and mediated immunoassays, such as ELISAs; biotin/avidin type assays; radioimmunoassays; immunoelectrophoresis; immunoprecipitation, capillary electrophoresis-mass spectroscopy technique (CE-MS) etc. The reactions generally include revealing labels such as fluorescent, chemioluminescent, radioactive, enzymatic labels or dye molecules, or other methods for detecting the formation of a complex between the antigen and the antibody or antibodies reacted therewith. The aforementioned assays generally involve separation of unbound protein in a liquid phase from a solid phase support to which antigen-antibody complexes are bound. Solid supports which can be used in the practice of the invention include substrates such as nitrocellulose (e.g., in membrane or microtiter well form); polyvinylchloride (e.g., sheets or microtiter wells); polystyrene latex (e.g., beads or microtiter plates); polyvinylidine fluoride; diazotized paper; nylon membranes; activated beads, magnetically responsive beads, and the like. More particularly, an ELISA method can be used, wherein the wells of a microtiter plate are coated with a set of antibodies against the proteins to be tested. A sample containing or suspected of containing the marker protein is then added to the coated wells. After a period of incubation sufficient to allow the formation of antibody-antigen complexes, the plate(s) can be washed to remove unbound moieties and a detectably labelled secondary binding molecule is added. The secondary binding molecule is allowed to react with any captured sample marker protein, the plate is washed and the presence of the secondary binding molecule is detected using methods well known in the art. Methods of the invention may comprise a step consisting of comparing the proteins and fragments concentration in circulating cells with a control value. As used herein, “concentration of protein” refers to an amount or a concentration of a transcription product, for instance the proteins int-FH. Typically, a level of a protein can be expressed as nanograms per microgram of tissue or nanograms per milliliter of a culture medium, for example. Alternatively, relative units can be employed to describe a concentration. In a particular embodiment, “concentration of proteins” may refer to fragments of the protein int-FH. Thus, in a particular embodiment, fragment of int-FH protein may also be measured.

Predetermined reference values used for comparison of the expression levels may comprise “cut-off” or “threshold” values that may be determined as described herein. Each reference (“cut-off”) value for int-FH level may be predetermined by carrying out a method comprising the steps of
a) providing a collection of samples from patients suffering of a cancer;
b) determining the level of int-FH for each sample contained in the collection provided at step a);
c) ranking the tumor tissue samples according to said level
d) classifying said samples in pairs of subsets of increasing, respectively decreasing, number of members ranked according to their expression level,
e) providing, for each sample provided at step a), information relating to the actual clinical outcome for the corresponding cancer patient;
f) for each pair of subsets of samples, obtaining a Kaplan Meier percentage of survival curve;
g) for each pair of subsets of samples calculating the statistical significance (p value) between both subsets
h) selecting as reference value for the level, the value of level for which the p value is the smallest.

For example, the expression level of int-FH has been assessed for 100 cancer samples of 100 patients. The 100 samples are ranked according to their expression level. Sample 1 has the best expression level and sample 100 has the worst expression level. A first grouping provides two subsets: on one side sample Nr 1 and on the other side the 99 other samples. The next grouping provides on one side samples 1 and 2 and on the other side the 98 remaining samples etc., until the last grouping: on one side samples 1 to 99 and on the other side sample Nr 100. According to the information relating to the actual clinical outcome for the corresponding cancer patient, Kaplan Meier curves are prepared for each of the 99 groups of two subsets. Also for each of the 99 groups, the p value between both subsets was calculated.

The reference value is selected such as the discrimination based on the criterion of the minimum p value is the strongest. In other terms, the expression level corresponding to the boundary between both subsets for which the p value is minimum is considered as the reference value. It should be noted that the reference value is not necessarily the median value of expression levels.

In routine work, the reference value (cut-off value) may be used in the present method to discriminate cancer samples and therefore the corresponding patients.

Kaplan-Meier curves of percentage of survival as a function of time are commonly used to measure the fraction of patients living for a certain amount of time after treatment and are well known by the man skilled in the art.

The man skilled in the art also understands that the same technique of assessment of the expression level of a protein should of course be used for obtaining the reference value and thereafter for assessment of the expression level of a protein of a patient subjected to the method of the invention.

Inhibitor of int-FH

The inventors have shown that an inhibition of int-FH by siRNAs influence proliferation, cell cycle, viability and morphology of lung adenocarcinoma or clear cell renal cell carcinoma. They observed cancer cells blocked in the G0/G1 phase and a downregulation of genes implicated in the G1/S transition of the mitotic cycle. Proliferation capacity of cancer cells was significantly reduced and migration capacity was also decreased, demonstrating that siFH was an efficient inhibitor of int-FH in lung adenocarcinoma or clear cell renal cell carcinoma.

Thus, another aspect of the present invention relates to a method for treating a cancer with an inhibitor of int-FH in a patient with a bad prognosis according to the method of the invention. In some embodiments, the cancer is a lung adenocarcinoma or a renal carcinoma. In some embodiments, the renal carcinoma is a clear cell renal cell carcinoma. Thus, in some embodiments, the cancer is a lung adenocarcinoma or a clear cell renal cell carcinoma. In some embodiments, the present invention relates to a method for treating a lung adenocarcinoma or a clear cell renal cell carcinoma with an inhibitor of int-FH in a patient with a bad prognosis according to the invention, wherein said inhibitor is a siFH. In some embodiments, the present invention relates to a method for treating a lung adenocarcinoma or a clear cell renal cell carcinoma with an inhibitor of int-FH in a patient with a bad prognosis according to the invention, wherein said inhibitor is a single domain antibody directed against int-FH.

As used herein, the term “treating” is defined as an approach for obtaining beneficial or desired results including clinical results. For purpose of this invention, beneficial or desired clinical results include, but are not limited to, one or more of the following: alleviating one or more symptoms resulting from disease, diminishing the extent of the disease, stabilizing the disease (e.g., preventing or delaying the worsening of the disease), preventing or delaying the spread of the disease, preventing or delaying the recurrence of the disease, delaying or slowing the progression of the disease, ameliorating the disease state, providing a remission (partial or total) of the disease, decreasing the dose of one or more other medications required to treat the disease, delaying the progression of the disease, increasing the quality of life and/or prolonging survival. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein, the terms “administering” or “administration” refer to the act of injecting or otherwise physically delivering a substance as it exists outside the body (e.g., an inhibitor of int-FH) into the subject, such as by mucosal, intradermal, intravenous, subcutaneous, intramuscular delivery and/or any other method of physical delivery described herein or known in the art. When a disease, or a symptom thereof, is being treated, administration of the substance typically occurs after the onset of the disease or symptoms thereof. When a disease or symptoms thereof, are being prevented, administration of the substance typically occurs before the onset of the disease or symptoms thereof.

As used herein, the expression “therapeutically effective amount” is meant a sufficient amount of the active ingredient (e.g. an inhibitor of int-FH) for treating or reducing the symptoms at reasonable benefit/risk ratio applicable to any medical treatment. It will be understood that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination with the active ingredients; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, typically from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

As used herein, the term “inhibitor of int-FH” refers to molecules or compound which can inhibit the activity of the proteins (e.g. interaction with its partner molecules, including but not limited to C3 activation fragments, as found in the lysosomes) or a molecule or compound which destabilizes the protein. In a particular embodiment, an inhibitor of int-FH can inhibit the expression of CFH CFHL-1 gene coding for the protein to decrease its pro tumoral action in a patient with a bad prognosis. An inhibitor of int-FH can also isolate and neutralize the internal FH to increase survival rate. In some embodiments, said inhibitor is a siFH. In some embodiments, said inhibitor is a single chain antibody directed against int-FH.

In order to test the functionality of a putative int-FH inhibitor, a test is necessary. For that purpose, to identify int-FH inhibitors, the cancer cell lines (A498, Caki-1, A549 or another, which changes phenotype upon FH silencing) should be exposed to increasing concentration of the candidate inhibitor(s). The alteration of morphology, proliferation, survival and migration by the inhibitor has to be compared to vehicle or irrelevant molecule-treated cells. Cells silenced for FH should be used as positive control.

In one embodiment, the inhibitors according to the invention may be a low molecular weight compound, e.g. a small organic molecule (natural or not).

The term “small organic molecule” refers to a molecule (natural or not) of a size comparable to those organic molecules generally used in pharmaceuticals. The term excludes biological macromolecules (e.g., proteins, nucleic acids, etc.). Preferred small organic molecules range in size up to about 10000 Da, more preferably up to 5000 Da, more preferably up to 2000 Da and most preferably up to about 1000 Da.

In one embodiment, the int-FH inhibitor according to the invention is an inhibitor of the int-FH gene expression.

Int-FH gene expression can be reduced by contacting a subject or cell with a small double stranded RNA (dsRNA), or a vector or construct causing the production of a small double stranded RNA, such that int-FH gene expression is specifically inhibited (i.e. RNA interference or RNAi). Methods for selecting an appropriate dsRNA or dsRNA-encoding vector are well known in the art for genes whose sequence is known (e.g. see for example Tuschl, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and International Patent Publication Nos. WO 01/36646, WO 99/32619, and WO 01/68836).

Ribozymes can also function as inhibitors of int-FH gene expression for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of int-FH mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Both antisense oligonucleotides and ribozymes useful as inhibitors of int-FH gene expression can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Antisense oligonucleotides, siRNAs and ribozymes of the invention may be delivered in vivo alone or in association with a vector. In its broadest sense, a “vector” is any vehicle capable of facilitating the transfer of the antisense oligonucleotide siRNA or ribozyme nucleic acid to the cells and preferably cells expressing int-FH, like tumor cells. Preferably, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide siRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rouse sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

Preferred viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in Kriegler, 1990 and in Murry, 1991.

Preferred viruses for certain applications are the adeno-viruses and adeno-associated viruses, which are double-stranded DNA viruses that have already been approved for human use in gene therapy. The adeno-associated virus can be engineered to be replication deficient and is capable of infecting a wide range of cell types and species. It further has advantages such as, heat and lipid solvent stability; high transduction frequencies in cells of diverse lineages, including hemopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other vectors include plasmid vectors. Plasmid vectors have been extensively described in the art and are well known to those of skill in the art. See e.g. Sambrook et al., 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, eye, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

In a particular embodiment, the antisense oligonucleotide, siRNA, shRNA or ribozyme nucleic acid sequence is under the control of a heterologous regulatory region, e.g., a heterologous promoter. The promoter may be specific for Muller glial cells, microglia cells, endothelial cells, pericyte cells and astrocytes. For example, a specific expression in Muller glial cells may be obtained through the promoter of the glutamine synthetase gene is suitable.

The promoter can also be, e.g., a viral promoter, such as CMV promoter or any synthetic promoters.

As used herein, the term “siFH” refers to a small inhibitory RNA (siRNA) which reduce totally or partially the expression of int-FH by CFH CFHL-1 gene (Entrez Gene ID number: 3075; mRNA sequences references RefSeq: NM_000186.4). SiFH can function as inhibitors of CFH/CFHL-1 expression for use in the present invention. As example, a mixture of two siRNAs comprising Qiagen SI00003983 and SI00003990 siRNAs against CFH CFHL-1 genes is efficient.

As used herein, the term “single domain antibody” (sdAb) or “VHH” refers to the single heavy chain variable domain of antibodies of the type that can be found in Camelid mammals which are naturally devoid of light chains. Such VHH are also called “nanobody®”. According to the invention, sdAb can particularly be llama sdAb. The term “VHH” refers to the single heavy chain having 3 complementarity determining regions (CDRs): CDR1, CDR2 and CDR3. The term “complementarity determining region” or “CDR” refers to the hypervariable amino acid sequences which define the binding affinity and specificity of the VHH.

The VHH according to the invention can readily be prepared by an ordinarily skilled artisan using routine experimentation. The VHH variants and modified form thereof may be produced under any known technique in the art such as in-vitro maturation.

VHHs or sdAbs are usually generated by PCR cloning of the V-domain repertoire from blood, lymph node, or spleen cDNA obtained from immunized animals into a phage display vector, such as pHEN2. Antigen-specific VHHs are commonly selected by panning phage libraries on immobilized antigen, e.g., antigen coated onto the plastic surface of a test tube, biotinylated antigens immobilized on streptavidin beads, or membrane proteins expressed on the surface of cells. However, such VHHs often show lower affinities for their antigen than VHHs derived from animals that have received several immunizations. The high affinity of VHHs from immune libraries is attributed to the natural selection of variant VHHs during clonal expansion of B-cells in the lymphoid organs of immunized animals. The affinity of VHHs from non-immune libraries can often be improved by mimicking this strategy in vitro, i.e., by site directed mutagenesis of the CDR regions and further rounds of panning on immobilized antigen under conditions of increased stringency (higher temperature, high or low salt concentration, high or low pH, and low antigen concentrations). VHHs derived from camelid are readily expressed in and purified from the E. coli periplasm at much higher levels than the corresponding domains of conventional antibodies. VHHs generally display high solubility and stability and can also be readily produced in yeast, plant, and mammalian cells. For example, the “Hamers patents” describe methods and techniques for generating VHH against any desired target (see for example U.S. Pat. Nos. 5,800,988; 5,874,541 and 6,015,695). The “Hamers patents” more particularly describe production of VHHs in bacterial hosts such as E. coli (see for example U.S. Pat. No. 6,765,087) and in lower eukaryotic hosts such as moulds (for example Aspergillus or Trichoderma) or in yeast (for example Saccharomyces, Kluyveromyces, Hansenula or Pichia) (see for example U.S. Pat. No. 6,838,254).

Pharmaceutical Composition

The present invention also relates to a pharmaceutical composition comprising an inhibitor of int-FH for use in the treatment of a cancer, in a patient with a bad prognosis according to the method of the invention. In some embodiments, the cancer is a lung adenocarcinoma, a renal carcinoma or a liver cancer. In some embodiments, the cancer is a lung adenocarcinoma or a renal carcinoma. In some embodiments, the renal adenocarcinoma is a clear cell renal cell carcinoma. Thus, in some embodiments, the cancer is a lung adenocarcinoma or a clear cell renal cell carcinoma. Another object of the invention relates to a pharmaceutical composition comprising an inhibitor of int-FH for use in the treatment of a lung adenocarcinoma or a clear cell renal cell carcinoma, in a patient with a bad prognosis according to the method of the invention.

Any therapeutic agent of the invention may be combined with pharmaceutically acceptable excipients, and optionally sustained-release matrices, such as biodegradable polymers, to form therapeutic compositions.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The form of the pharmaceutical compositions, the route of administration, the dosage and the regimen naturally depend upon the condition to be treated, the severity of the illness, the age, weight, and sex of the patient, etc. The pharmaceutical compositions of the invention can be formulated for a topical, oral, intranasal, parenteral, intraocular, intravenous, intramuscular, intrathecal or subcutaneous administration and the like. Particularly, the pharmaceutical compositions contain vehicles which are pharmaceutically acceptable for a formulation capable of being injected. In some embodiments, the pharmaceutical composition contains at least one vehicle which is pharmaceutically acceptable to be injected directly in tumor. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. In a particular embodiment, the at least one vehicle is a liposome. As referenced in Ostro and Cullis (1989), liposomes can encapsulate water-soluble drugs in their aqueous spaces, but also lipid-soluble drugs within the membrane. Liposomes then deliver their drug content by interacting with cells. As example, many liposomes have been engineered to improve drug delivery such as described in EP 2 773 426 and EP 1 746 976. In a more particular embodiment, the at least one liposome is a targeting liposome in order to prevent side effects. Such targeting liposomes are, as example, described in EP 2 173 073. The doses used for the administration can be adapted as a function of various parameters, and in particular as a function of the mode of administration used, of the relevant pathology, or alternatively of the desired duration of treatment. In addition, other pharmaceutically acceptable forms include, e.g. tablets or other solids for oral administration; time release capsules; and any other form currently can be used.

Pharmaceutical compositions of the present invention may comprise a further therapeutic active agent. The present invention also relates to a kit comprising an agonist, antagonist or inhibitor of the expression according to the invention and a further therapeutic active agent.

For example, anti-cancer agents may be added to the pharmaceutical composition as described below.

Anti-cancer agents may be Melphalan, Vincristine (Oncovin), Cyclophosphamide (Cytoxan), Etoposide (VP-16), Doxorubicin (Adriamycin), Liposomal doxorubicin (Doxil) and Bendamustine (Treanda).

Others anti-cancer agents may be for example cytarabine, anthracyclines, fludarabine, gemcitabine, capecitabine, methotrexate, taxol, taxotere, mercaptopurine, thioguanine, hydroxyurea, cyclophosphamide, ifosfamide, nitrosoureas, platinum complexes such as cisplatin, carboplatin and oxaliplatin, mitomycin, dacarbazine, procarbizine, etoposide, teniposide, campathecins, bleomycin, doxorubicin, idarubicin, daunorubicin, dactinomycin, plicamycin, mitoxantrone, L-asparaginase, doxorubicin, epimbicm, 5-fluorouracil, taxanes such as docetaxel and paclitaxel, leucovorin, levamisole, irinotecan, estramustine, etoposide, nitrogen mustards, BCNU, nitrosoureas such as carmustme and lomustine, vinca alkaloids such as vinblastine, vincristine and vinorelbine, imatimb mesylate, hexamethyhnelamine, topotecan, kinase inhibitors, phosphatase inhibitors, ATPase inhibitors, tyrphostins, protease inhibitors, inhibitors herbimycm A, genistein, erbstatin, and lavendustin A. In one embodiment, additional anticancer agents may be selected from, but are not limited to, one or a combination of the following class of agents: alkylating agents, plant alkaloids, DNA topoisomerase inhibitors, anti-folates, pyrimidine analogs, purine analogs, DNA antimetabolites, taxanes, podophyllotoxin, hormonal therapies, retinoids, photosensitizers or photodynamic therapies, angiogenesis inhibitors, antimitotic agents, isoprenylation inhibitors, cell cycle inhibitors, actinomycins, bleomycins, MDR inhibitors and Ca2+ ATPase inhibitors.

Additional anti-cancer agents may be selected from, but are not limited to, cytokines, chemokines, growth factors, growth inhibitory factors, hormones, soluble receptors, decoy receptors, monoclonal or polyclonal antibodies, mono-specific, bi-specific or multi-specific antibodies, monobodies, polybodies.

Additional anti-cancer agent may be selected from, but are not limited to, growth or hematopoietic factors such as erythropoietin and thrombopoietin, and growth factor mimetics thereof.

In the present methods for treating cancer the further therapeutic active agent can be an antiemetic agent. Suitable antiemetic agents include, but are not limited to, metoclopromide, domperidone, prochlorperazine, promethazine, chlorpromazine, trimethobenzamide, ondansetron, granisetron, hydroxyzine, acethylleucine monoemanolamine, alizapride, azasetron, benzquinamide, bietanautine, bromopride, buclizine, clebopride, cyclizine, dunenhydrinate, diphenidol, dolasetron, meclizme, methallatal, metopimazine, nabilone, oxypemdyl, pipamazine, scopolamine, sulpiride, tetrahydrocannabinols, thiefhylperazine, thioproperazine and tropisetron. In a preferred embodiment, the antiemetic agent is granisetron or ondansetron.

In another embodiment, the further therapeutic active agent can be a hematopoietic colony stimulating factor. Suitable hematopoietic colony stimulating factors include, but are not limited to, filgrastim, sargramostim, molgramostim and epoietin alpha.

In still another embodiment, the other therapeutic active agent can be an opioid or non-opioid analgesic agent. Suitable opioid analgesic agents include, but are not limited to, morphine, heroin, hydromorphone, hydrocodone, oxymorphone, oxycodone, metopon, apomorphine, nomioiphine, etoipbine, buprenorphine, mepeddine, lopermide, anileddine, ethoheptazine, piminidine, betaprodine, diphenoxylate, fentanil, sufentanil, alfentanil, remifentanil, levorphanol, dextromethorphan, phenazodne, pemazocine, cyclazocine, methadone, isomethadone and propoxyphene. Suitable non-opioid analgesic agents include, but are not limited to, aspirin, celecoxib, rofecoxib, diclofinac, diflusinal, etodolac, fenoprofen, flurbiprofen, ibuprofen, ketoprofen, indomethacin, ketorolac, meclofenamate, mefanamic acid, nabumetone, naproxen, piroxicam and sulindac.

In yet another embodiment, the further therapeutic active agent can be an anxiolytic agent. Suitable anxiolytic agents include, but are not limited to, buspirone, and benzodiazepines such as diazepam, lorazepam, oxazapam, chlorazepate, clonazepam, chlordiazepoxide and alprazolam.

In yet another embodiment, the further therapeutic active agent can be a checkpoint blockade cancer immunotherapy agent.

Typically, the checkpoint blockade cancer immunotherapy agent is an agent which blocks an immunosuppressive receptor expressed by activated T lymphocytes, such as cytotoxic T lymphocyte-associated protein 4 (CTLA4) and programmed cell death 1 (PDCD1, best known as PD-1), or by NK cells, like various members of the killer cell immunoglobulin-like receptor (KIR) family, or an agent which blocks the principal ligands of these receptors, such as PD-1 ligand CD274 (best known as PD-L1 or B7-H1).

Typically, the checkpoint blockade cancer immunotherapy agent is an antibody.

In some embodiments, the checkpoint blockade cancer immunotherapy agent is an antibody selected from the group consisting of anti-CTLA4 antibodies, anti-PD1 antibodies, anti-PDL1 antibodies, anti-PDL2 antibodies, anti-TIM-3 antibodies, anti-LAG3 antibodies, anti-IDO1 antibodies, anti-TIGIT antibodies, anti-B7H3 antibodies, anti-B7H4 antibodies, anti-BTLA antibodies, and anti-B7H6 antibodies.

The invention will be further illustrated by the following figures and examples. However, these examples and figures should not be interpreted in any way as limiting the scope of the present invention.

### FIGURES

FIG. 1: Int-FH produced by tumor cells but not ext-FH is associated with poor prognosis in ccRCC. A) Kaplan-Meier curve of DFS according to the high/low classification for FH deposits (ext-FH) in ccRCC cohort 1. A log-rank test was used, and p<0.05 was considered significant. B-C) Kaplan-Meier curves of PFS and OS according to the high/low classification for FH deposits (ext-FH) in ccRCC cohort 2. A log-rank test was used, and p<0.05 was considered significant. D) Kaplan-Meier curve of DFS according to the high/low classification for int-FH in ccRCC cohort 1. A log-rank test was used, and p<0.05 was considered significant. E-F) Kaplan-Meier curves of PFS and OS according to the high/low classification for int-FH in ccRCC cohort 2. A log-rank test was used, and p<0.05 was considered significant.

FIG. 2: FH silencing modifies ccRCC tumor cell phenotype. Evaluation of proliferation of the ccRCC A498 cells after FH silencing. A) Fold change in CFSE geometric means in A498 siFH vs. A498 siC cells in 4 independent experiments. B) Percentage of cells in each cell cycle phase (G0-G1/S/G2-M) determined by flow cytometry analysis of PI staining on A498 siFH or siC cells in four independent experiments. C) Evaluation of viability, measured by the percentage of DAPI-positive dead cells determined by flow cytometry after 72 h of culture in A498 siFH or A498 siC cells in five independent experiments. D) An ImageJ wound healing macro was used to determine the area of the scratch at each time point. The percentage of closing of the scratch area was calculated with the following calculation: 100-(scratch area TO/scratch area t+12 h).

FIG. 3: Int-FH but not ext-FH exerts a protumoral effect on lung adenocarcinoma. A) FH concentration in the supernatant of A549 cells (siFH, siC, WT) determined by ELISA. FH produced by A549 cells regulates complement.

Ext-FH has no impact on tumor growth in a mouse model of lung cancer. B) The quantification of the FH staining surface was determined by HALO software for 4 WT mice and 4 hepato−/− mice. The boxplot represents the ratio between the FH staining area and the total tissue area for each mouse.

FIG. 4: Int-FH is associated with decreased survival of patients with lung ADK. Kaplan-Meier curves for OS according to the high/low classification for int-FH in NSCLC cohort 3. A) Lung ADK. B) Lung SCC. A log-rank test was used, and p<0.05 was considered significant.

### EXAMPLE

Material & Methods

Patients

Primary tumor specimens were collected from 2 retrospective cohorts of ccRCC patients and one retrospective cohorts of non-small cell lung patients. Plasma samples were collected from a prospective cohort of ccRCC.

Animal Experimentation

400000 mouse TC-1 tumor cells were s.c. inoculated in the right flank with 200 μl PBS. Tumor size was measured with calipers every 2-3 days for 25 days or until reaching 3000 mm3. Two independent experiments were performed with 5 WT and 5 hepatoFH−/−mice aged 8-10 weeks (experiment 1) and with 10 WT and 5 hepatoFH−/−mice (experiment 2).

Immunofluorescence (IF)/Immunohistochemistry (IHC)

Formalin-fixed paraffin-embedded (FFPE) human tumor specimens of RCC or NSCLC were stained for FH, C3d, cytokeratin, αSMA, CD31, CD163, CD20, CD3, N-cadherin, E-cadherin, vimentin, (Supplementary Table 4). Antigen retrieval was performed with PT-link (Dako) using EnVision FLEX Retrieval Solutions (Dako) with high or low pH depending on the antibody. The endogenous peroxidases were then blocked by using 3% H₂O₂(Gifrer) solution, followed by blocking of nonspecific staining with Protein Block (Dako). The staining was revealed by 3-amino-9-ethylcarbazole substrate (Vector Laboratories). The slides were then counterstained with hematoxylin (Dako), mounted with Glycergel (Dako) and scanned with NanoZoomer (Hamamastu). For double staining by IF, a tyramide system was used to avoid species compatibility problems and to increase the fluorescence signal. This system needed a supplementary step after the secondary HRP antibody that consisted of an incubation with AF647 tyramide reagent (diluted 1:100 in TBS 1X, H₂O₂ 0.0015%, Life Technologies). The entire staining protocol was then repeated for the second primary antibody but with AF546 tyramide reagent (Life Technologies). The nuclei were stained with DAPI. The slides are then mounted with Prolong Glass antifade reagent and scanned with AxioScan (Zeiss). The colocalization between FH and CK was analyzed by using HALO Image Analysis software (Indica Labs).

Patient stratification. For patient stratification, the tumors were classified into 2 groups, high or low, by a semiquantification method to distinguish the different types of FH staining: int-FH or extracellular (ext-FH). For FH int-FH or ext-FH staining, the high group corresponded to a number of FH-positive cells >30% and the low group corresponded to that <30%. Three independent observers (MVD, MR, RT or LTR) performed the analysis to avoid any bias. Automated quantification was not feasible since it was not possible to train the algorithm to reliably distinguish intracellular staining from membrane deposits.

Mouse tumor staining. 6 μm sections of frozen mouse tumor tissues were fixed with cold acetone for 8 minutes and blocked with 5% BSA-TBS for 30 minutes. Tumor sections were then stained with a rabbit polyclonal anti-CD31 (Abcam, ab124432) or a goat polyclonal antiserum anti-FH (Quidel, A312) as primary antibodies and goat anti-rabbit coupled to AF647 (Thermo Fisher Scientific, A-21245) or donkey anti-goat coupled to AF647 (Thermo Fisher Scientific, A-21447) as secondary antibodies.

Cell Lines

Human ccRCC cell lines (Caki-1 and A498), human lung adenocarcinoma (A549) and squamous cell carcinoma (SK-MES) cell lines, primary human umbilical vein endothelial cells (HUVECs), primary renal proximal tubule cells (RPTECs) and mouse lung cancer TC-1 were used in this study.

Silencing of CFH

The cells were transfected with a preprepared mixture of two siRNAs against CFH CFHL-1 at a concentration of 50 nM (Qiagen Hs_CFH_3_Flexitube siRNA SI00003983 and Hs_CFH_4_Flexitube siRNA SI00003990) or siRNA control 50 nM (Qiagen AllStars Negative Control siRNA, SI03650318), 5 μL of Lipofectamine RNAiMAX Transfection Reagent (Thermo Fisher, 13778030) in Opti-MEM medium (Gibco). After 24 h, the transfection was stopped by changing the Opti-MEM medium to the appropriate medium without antibiotics. The cells were ready to use for the functional experiments 72 h posttransfection. Photos were taken under a microscope 72 h posttransfection to determine the differences in cell number and morphology. The efficiency of the silencing was attested by RT-qPCR and ELISA for cell lysates and supernatants.

Protein Extraction

Cells were lysed in RIPA buffer to obtain a total cell lysate. A specific cell fractionation kit (Abcam, ab109719) was also used to prepare the organelles and cytoplasmic and nuclear fractions from cultured cells according to the manufacturer's instructions.

ELISA

FH concentrations in cell supernatants and cell lysates were measured by a homemade sandwich ELISA method by using a polyclonal antibody against FH (Calbiochem) under native state or biotinylated⁵⁴. The native anti-FH was coated on a Nunc MaxiSorp ELISA 96-well and 1% PBS-BSA was used for the blocking. The supernatant and cell lysate were added to the plate and incubated for one hour at RT. After washing, the plate was incubated with an in-house biotinylated anti-FH antibody for 1 h at RT. After additional washes, streptavidin coupled with horseradish peroxidase (HRP) (Dako, P039701-2) was added for 1 h at RT. The FH concentration was revealed by SureBlue TMB Microwell Peroxidase Substrate (KPL), and the reaction was stopped by 2 mol/L sulfuric acid. Multiskan Ex (Thermo Fisher Scientific) was used to read the optical density at 450 nm. The results are expressed in μg/mL according to the standard curve, made using commercial purified FH (Comptech).

Western Blot

The supernatants of cells cultured without serum for 48 h were recovered and concentrated using Amicon Ultracel 3K units (UFC, 900324). A 10% Bis-Tris gel (Thermo Fisher Scientific) was used to separate the proteins before transfer onto a nitrocellulose membrane. After 1 h of blocking using 5% BSA-TBS, the membrane was incubated overnight at 4° C. on a rocking platform with the primary antibodies (supplementary table 5). Purified C3 (Calbiochem, 204885), purified C3b (Calbiochem, 204860) or purified iC3b (Calbiochem, 204863) were used as controls.

RT-qPCR

RNA was extracted from the cultured cell lines or from sections of mouse tumors using a Maxwell cell 16 LEV simplyRNA Purification Kit (Promega, AS1270). The quality and quantity of RNA were determined by a 2100 Bioanalyzer (Agilent) using an Agilent RNA 6000 Nano Assay kit (5067-1511). An Applied Biosystem High-capacity cDNA Reverse Transcription Kit (Applied Biosystem, 4368814) was used for reverse transcription.

RNA-seq

The RNA of 3 independent biological replicates for each condition (siFH vs siC) was used for the RNA-seq study. Sequencing was then carried out on a paired-end 75 bp Illumina HiSeq 4000 instrument. The differential expression analysis between siFH and siC was then performed by using R software and the DESeq2 package⁵⁵.

Flow Cytometry

C3 deposits. Normal human serum diluted to 33% in PBS was added to the cells for 30 minutes at 37° C. with or without an anti-FH blocking antibody (hybridoma, Ox-24). Staining was performed with mouse monoclonal anti-C3c (Quidel, A205) or isotype control IgG1 followed by a secondary goat anti-mouse AF700 antibody (Invitrogen, A-21036).

Proliferation and viability. CFSE reagent 1:1000 (Invitrogen, C34554) was adding to the cells for 20 minutes at 37° C. Complete culture medium was added to stop CFSE staining. Cells were seeded in a 6-well plate and cultured for 72 h in the presence or absence of complete medium supplemented with purified FH 2 μg/mL or 20 μg/mL. The proliferation capacity of cells was also evaluated after the addition of either 50% complete medium and 50% A498 supernatant after 72 h of culture. Supernatants containing dead cells and adherent cells were recovered and stained with DAPI before analysis by flow cytometry.

Cell cycle. After fixation in cold 70% ethanol, 3 μM propidium iodide (PI, Invitrogen, P1304MP) was added to the cells in the presence of RNase for 15 minutes at RT.

FH staining. For int-FH staining, after blocking with 5% BSA, cells were stained with DAPI (1:1000) for 10 minutes at RT. The cells were then permeabilized with a BD Cytofix/Cytoperm kit (BD, 554714). An anti-FH antibody 1:100 (Quidel, A224) or isotype control was added to cells for 30 minutes at RT. A goat anti-mouse AF488 antibody (Thermo Fisher, A28175) diluted 1:100 was adding after washing.

Immunocytofluorescence

Cells were seeded in a round cover glass and after one day of adherence were fixed with 4% PFA for 30 minutes at RT and stained for their actin cytoskeleton with Phalloidin-IFluor 488 reagent (Abcam, ab176753), and the nuclei were stained with DAPI.

Migration

When 90% cell confluence was reached, a wound on the cell monolayer was created with a pipette tip. Photos of the scratch were taken under a microscope just after the scratch and 12 h later. The scratch area was determined with ImageJ using the macro wound healing tool (http://dev.mri.cnrs.fr/projects/imagej-macros/wiki/Wound_Healing_Tool) to calculate the recovery percentage.

Results

Int-FH but not ext-FH is associated with a negative impact on ccRCC patient survival

In the TCGA database, in the ccRCC cohort (KIRC cohort), high gene expression of CFH, above the median, was significantly associated with decreased DFS (p=0.03) and OS (p=0.017), suggesting a role of locally produced FH in this type of cancer.

Staining for FH of ccRCC tumor sections revealed heterogeneous staining patterns by immunohistochemistry (IHC), represented by deposits, defined as membranous staining, surrounding the tumor cell, named here extracellular FH (ext-FH) and an intracellular staining of the tumor cells (int-FH) (data not shown). Analysis of the intracellular staining revealed a diffuse coloration of the entire cytoplasm and/or intense granular staining, confirmed in consecutive sections using 4 different anti-FH monoclonal antibodies, recognizing different epitopes of the molecule (data not shown). The granular FH staining colocalized with lysosomal marker LAMP-1 but not with mitochondrial marker Tom20, endoplasmic reticulum marker calnexin, Golgi apparatus marker GOLGA5, peroxisome marker ABCD5 and lipid droplets marker PLIN2 or nuclear marker DAPI (data not shown).

The antibody Ox24 was selected for further use and its specificity for FH was validated by staining of liver sections and inhibition of the signal by pre-incubation with purified FH (data not shown). Within the tumor over ˜85% of the FH staining localized in tumor cells (cytokeratin) after automatic quantification, confirming the visual observation that tumor cells are the main ones that stain positive for FH (data not shown). To a lesser extent, stromal cells, especially fibroblasts, endothelial cells and a small fraction of macrophages, were also positive for FH, as attested by the colocalization of FH and αSMA, CD31 or some CD163+ cells, whereas there was no colocalization with CD20+ B cells and CD3+ T cells (data not shown). The plasma concentration of FH was not different between ccRCC patients and healthy donors, suggesting that FH production by the tumor itself contributes little, if at all, to the plasmatic pool (data not shown).

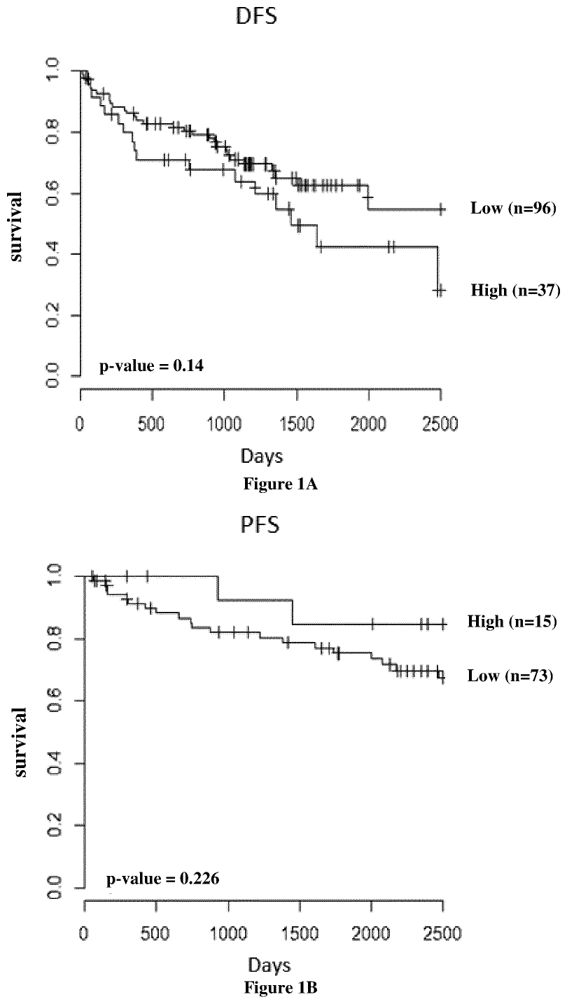

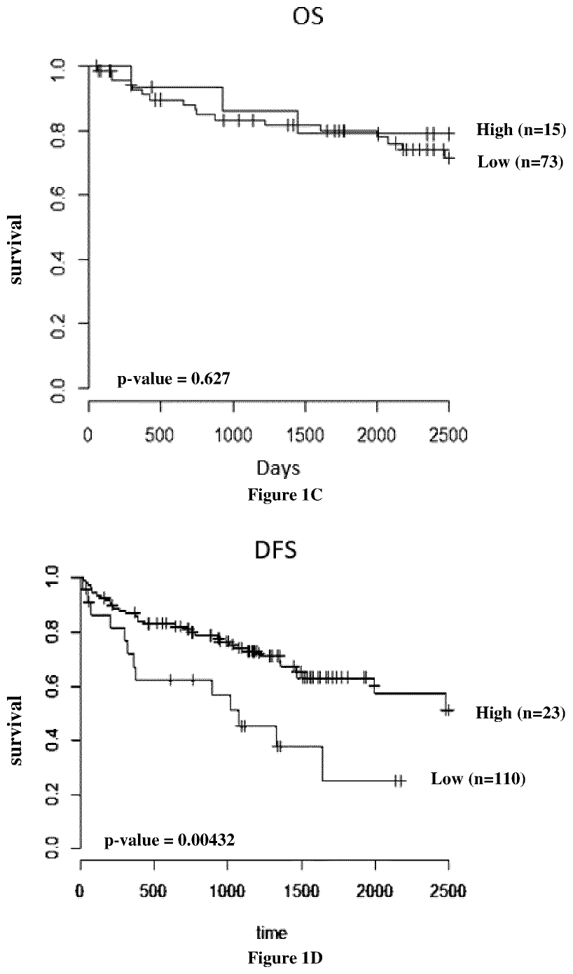

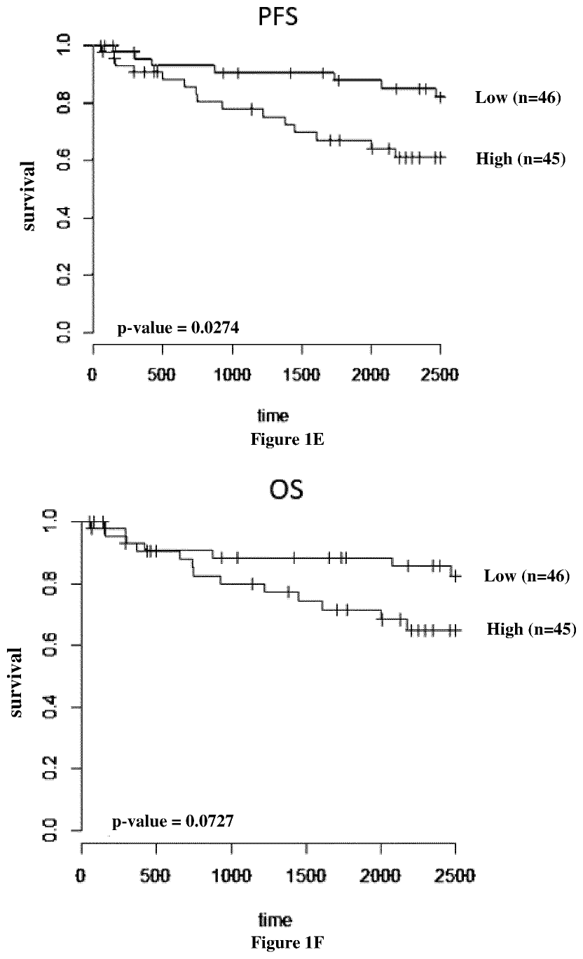

To have access to the specific impact of ext-FH and int-FH, we stained tumoral tissues from 2 cohorts of ccRCC patients by IHC. Ext-FH staining localized at the surface of tumor cells and was very heterogeneous between patients and inside the same tumor in the positive and negative areas. These staining patterns allowed us to classify the patients into two groups according to the number of tumor cells carrying FH deposits (data not shown). In the two cohorts of ccRCC, the presence of such deposits did not have any impact on survival (cohort 1, DFS, p=0.14; cohort 2, PFS, p=0.226 and OS p=0.627) (FIG. 1A, B, C). The same staining heterogeneity was observed for int-FH, and two groups of patients were constituted depending on the number of FH-producing cells (data not shown). Compared to the low density of int-FH-positive cells, a high density of int-FH-positive cells had a negative impact on the DFS of cohort 1 (p=0.00432) (FIG. 1D) and cohort 2 (PFS: p=0.0274, OS: p=0.0727) (FIG. 1E, F). The presence of FH inside the tumor cells correlated with tumor size and Fuhrman grade and with the other complement stainings, especially for intracellular C4 and C3. No association was found between int-FH and the immune infiltrate (data not shown).

Silencing of FH Modifies the Transcriptome of ccRCC Tumor Cells

Since FH was expressed mainly by tumor cells, we confirmed that ccRCC tumor cell lines A498 and Caki-1 also produce it (data not shown). Moreover, in both cell lines, FH was present both intracellularly, detected by flow cytometry and in the lysate, by western blot, and was secreted in the supernatant (data not shown). Therefore, we studied the impact of FH on key characteristics of tumor cells, using gene silencing. After silencing, the secretion of FH was reduced by 90-96% in the supernatant and 85-90% in the cell lysates for ccRCC lines A498 and Caki-1 (data not shown). The siRNA-induced silencing of FH revealed a high impact on the ccRCC tumor cell phenotype, which acquired a round shape. To identify the biological processes impacted by FH silencing, RNA-seq was performed on 3 biological replicates for each condition for the two ccRCC cell lines A498 and Caki-1 (siFH cells or siC cells). We used p<0.05 and log 2-fold change >1 to select the differentially expressed genes. With these criteria, in A498 cells, 235 were differentially regulated after FH silencing (data not shown). Among these genes, 139 were upregulated and 96 were downregulated. For Caki-1 cells, 192 genes were upregulated and 251 were downregulated after FH silencing (data not shown). The 50 most differentially expressed genes based on the adjusted p-value (padj) for the ccRCC cell lines siFH and siC are represented in the heatmap (data not shown). The functional classification analyses of all significantly up- and downregulated genes using the GO “Biological Pathway” category revealed that these genes were implicated in different biological processes (data not shown).

Silencing of FH Modifies the Phenotype of ccRCC Tumor Cells

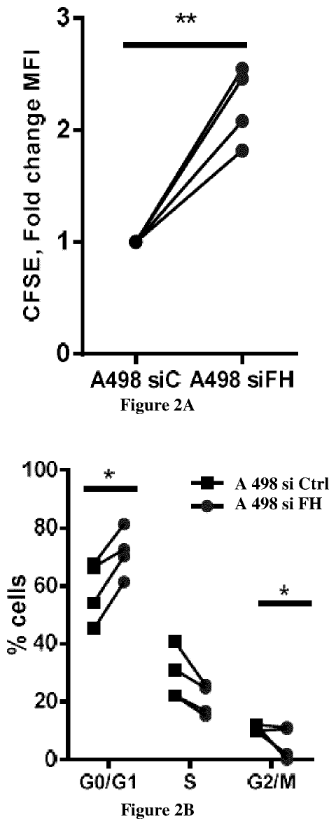

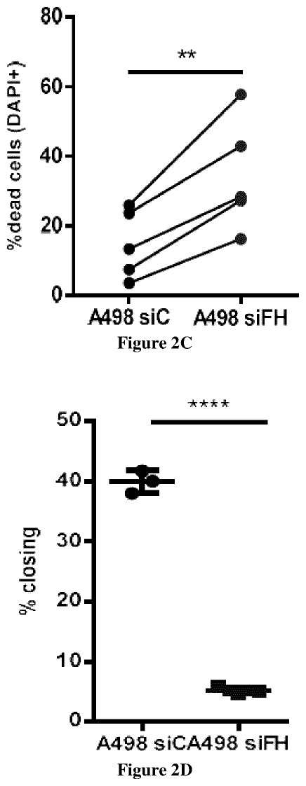

First, we observed an enrichment of genes implicated in cell proliferation in siFH cells and in parallel a downregulation of genes implicated in the G1/S transition of the mitotic cycle. We further confirmed that these pathways were considerably altered in FH-silenced cells and that the cell proliferation capacity of A498 ccRCC cells was significantly reduced (FIG. 2A). Importantly, addition of exogenous FH in the culture medium of A498 siC cells neither modify their ability to proliferate nor restored the basal proliferation level of the siFH cells (data not shown). Moreover, the addition of untreated A498 cell supernatant to siFH cells had no effect on their proliferation capacity (data not shown). These results show that exogenous FH, either purified from plasma or produced by the tumor cells themselves (which may differ from the plasma-derived FH), has no effect on tumor cell proliferation. Therefore, int-FH but not ext-FH is essential for the tumor cell phenotype.

Further, we found that the siFH blocked the cells in the G0/G1 phase (FIG. 2B), in parallel with the RNAseq data. Cell viability was also affected by an increase in the number of dead cells when tumor cells were deficient in FH (FIG. 2C). This could be explained by the increased p53 expression in A498 siFH cells, which is phosphorylated at the S46 and S15 positions (data not shown).

Moreover, some modifications of genes involved in cell adhesion, extracellular matrix organization and cell morphogenesis were also detected with RNA-seq analysis. Importantly, these transcriptional changes had functional repercussions with a strong modification of the actin cytoskeleton. Indeed, FH-silenced cells were rounder with fewer actin filaments and stress fibers (data not shown). Furthermore, there were also some differentially expressed genes that were involved in cell locomotion and cellular movement. In vitro, we confirmed that the migration capacity was also decreased with only 5% scratch recovery at 12 h for A498 siFH cells compared with 40% scratch area recovery for A498 siC cells (FIG. 2D). In situ, the more motile cells in a tumor are usually the ones undergoing epithelial to mesenchymal transition (EMT). On ccRCC sections, FH colocalized with those tumor cells, which presented with low levels of the epithelial marker cytokeratin (CK) (data not shown). An increased intensity of CK staining was associated with a decreased positivity for FH: cells with low intensity for CK represented 65% of FH+ cells, whereas tumor cells with medium and high intensity of CK represented only 15% and 5% of FH+ cells, respectively (data not shown). In addition, in ccRCC, FH-expressing cells also expressed the mesenchymal markers N-cadherin and vimentin (data not shown), suggesting that int-FH is associated with a mesenchymal and hence more motile tumor cell phenotype.

Finally, FH silencing resulted in downregulation of genes, related to cargo loading on vesicles. In line with this, in situ int-FH was located mainly in intracellular vesicles (data not shown). Moreover, by using successive detergent and centrifugation steps, and fractionation of the different cell compartments from the tumor cells, we found that the majority of FH protein was detected in a compartment containing organelle fraction (mitochondria/lysosomes/endoplasmic reticulum/vesicles), and very little FH was found in the cytosolic or nuclear compartment (data not shown). Using IF, we identified lysosomes as the organelles containing int-FH (data not shown). C3 is the main partner of FH in the extracellular compartment and has a lysosomal localization in T cells (20). By cell fractionation, we showed that C3 and FH were located in the same organelles (data not shown). Furthermore, IF staining with antibodies recognizing epitopes in C3d, C3c or C3a confirmed that FH and C3 were localized in the lysosomes, suggesting an intra-lysosomal interaction (data not shown). Both FH and C3 can be considered full-length proteins as the staining was detected with different antibodies that detect different doamins of FH and C3 (data not shown). Although C3 can be cleaved by cathepsin L (20) with the help of FH (34), we found that FH and cathepsin L did not colocalize in the ccRCC tumor sections with the limit of our detection method (data not shown). Nevertheless, we found colocalization of C3 with Factor B, indicating that a C3/C5 convertase can be formed intracellularly, especially in intracellular vesicles near the cell membrane (not shown), as in macrophages³¹. Therefore, C3 may be cleaved intracellularly to generate C3a. In turn C3a could signal via intracellular C3aR, activating mTOR pathways and regulating cell metabolism.

The exact molecular mechanisms by which FH impacts these biological processes is still unknown, but if could be at least in part related to interaction of the int-FH with int-C3, since partial colocalization was observed between these two proteins in the cytoplasmic vesicles, close to the nucleus. Using proximity ligation assay we confirmed the direct binding if int-FH and int-C3 (as well as their interaction in the context of deposits on the cell membrane, ext-FH with ext-C3). In some vesicles int-FH colocalized with int-C3 and int-Factor B, suggesting intracellular regulation of the C3/C5 convertase. Alternatively, int-FH could have an independent interactome, as in retinal pigment epithelial cells³².

Key findings on proliferation, cell cycle, viability, morphology and subcellular localization of FH were confirmed with the Caki-1 cell line (data not shown).

Ext-FH is Responsible for Complement Regulation but has No Impact on Cellular Function

Previous studies indicated that tumor cells produce FH, which can be used as a potential mechanism to escape complement attack³⁰. Ext-FH colocalized and correlated with the presence of C3d deposits at the surface of tumor cells, suggesting that FH plays its role as a complement regulator (data not shown). Compared to siC cells, when A498 or Caki-1 cells were silenced for FH and exposed to serum, an increased amount of C3 fragment deposits was observed (data not shown). Moreover, in the supernatant of A498 and Caki-1 siFH cells, the α43 band corresponding to the inactive fragment of C3b and C3(H₂O) (iC3b, iC3(H₂O)), was largely reduced due to a loss of factor I (FI) cofactor activity (data not shown). Indeed, factor I was secreted in the supernatants of ccRCC tumor cells (data not shown). Of note, the presence of C3b was detected in only Caki-1 cell supernatant (presence of α′ band), while both Caki-1 and A498 cells secreted C3/C3(H₂O) (presence of a band). The obtained results indicated that the silencing of FH results in a deregulation of the complement system and an increased level of C3 activation. To decipher whether this overactivation of complement can impact the tumor cell phenotype, we treated A498 cells with an FH blocking antibody in excess compared to the amount of FH secreted, Ox-24, at 20 μg/mL. The level of C3 deposits increased when cells were treated with this blocking antibody, reflecting its capacity to prevent FH binding to C3b and its regulatory function (data not shown). However, the tumor cell phenotype was not modified, contrary to the condition when cells were silenced for FH (data not shown), suggesting that extracellular complement regulatory functions had no impact in ccRCC tumor cells phenotype.

Finally, both A498 and Caki-1 express C3aR and C5aR1. Therefore, it is possible that the effect of tumor cells-produced FH is related to the control of the autocrine C3a and C5a, generated by the cells themselves. C5a in both untreated and concentrated supernatant was below the detection limit of the ELISA kit. C3a was detected at the limit of the ELISA only in the concentrated supernatant of Caki-1, while it was undetectable in A498. These results are in line with the pattern of the western blot, where the α′ band (corresponding to C3b and indicative of C3a generation) was detected only in the Caki-1 supernatant (data not shown). Therefore, even if FH may affect the local concentration of C3a and C5a in other contexts, this is not a likely mechanism in ccRCC.

The Biological Effect of FH is Specific for Tumor Cells

Since ccRCC is derived from proximal tubules, we tested FH silencing in this cell type. Proximal tubules stained positive for FH in the peritumoral tissue (data not shown). In primary human RPTECs, we detected FH in the lysate and the supernatant. FH was predominant in the same organelle fraction within these cells as within the tumor cells (data not shown). Despite the efficient FH silencing (validated by ELISA and Western blot on cell lysate or supernatant, data not shown), no modification of the transcriptomic program of the cells was observed with only 11 differentially expressed genes, including CFH, between siFH and siC RPTECs compared to 228 genes for A498 cells (data not shown). Moreover, only 2 changes were commonly differentially expressed in A498 cells and RPTECs: CFH and NCKAP5. No effects of FH silencing on the RPTECs phenotype were detected in terms of viability, number of cells or morphology. The actin cytoskeleton of the cells remained the same regardless of whether FH was silenced, and the migration capacity did not change (data not shown). Thus, the effect of FH seems to be specific to tumor cells.

Within the tumor, endothelial cells can also produce FH (data not shown). In vitro, endothelial cells (HUVECs) also produce and secrete FH. After efficient silencing, no alterations in the phenotype were found in terms of cell number, morphology, actin cytoskeleton and migration capacity (data not shown). The glomerular endothelial cells, in contrast, showed a phenotype upon FH knockdown (not shown), which was also dependent on the int-FH, as purified FH added to the culture medium could not rescue the phenotype (not shown), while another team showed that transfection with a plasmid coding for mouse FH rescued the phenotype of mouse FH-deficient cells³⁴.

Protumoral Effect of Int-FH in Lung Adenocarcinoma

The presence of int-FH positive cell inside the tumor is not specific for ccRCC since it was also found in NSCLC tumors (data not shown). However, Ext-FH was detected only rarely and in small areas of NSCLC tumors (data not shown). The main cell type staining positive for FH here again was the tumor cell as revealed by double staining with CK and especially by mesenchymal tumor cells that expressed high levels of N-cadherin and low levels of E-cadherin (data not shown). Moreover, stromal cells such as αSMA-positive fibroblasts and CD31-positive endothelial cells can also contribute to the local production of FH (data not shown). CD163+ macrophages, CD20+ B cells and CD3+ T cells did not stain positive for FH (data not shown).

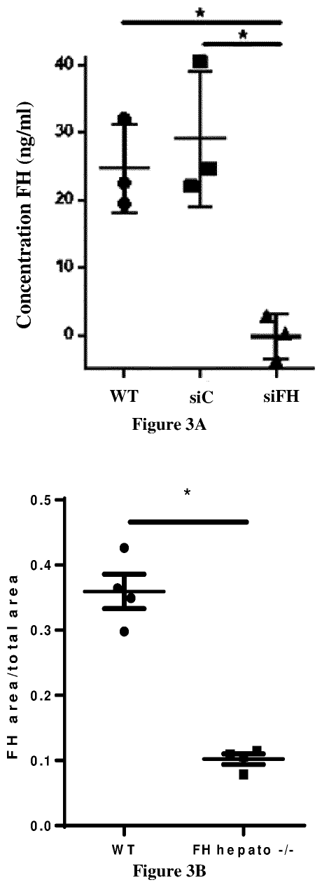

To determine whether the protumoral effect of int-FH could be relevant to NSCLC two cells lines have been selected to perform in vitro experiments; A549, a lung ADK tumor cell line and SK-MES, a lung SCC cell line. A549 cells produce and secrete equivalent amounts of FH as ccRCC cell lines (data not shown). The efficient silencing of FH (FIG. 3A) strongly modified the cell phenotype with differences in morphology and proliferation capacity, but without an effect on cell cycle (data not shown). In contrast, in a lung SCC cell line (SK-MES), no effects on the phenotype were detected despite efficient FH silencing (data not shown).

As expected, the efficient silencing of FH hampered the regulatory activity of FI, preventing C3(H2O) inactivation in the supernatant (data not shown). No α′ band indicative for C3b was detected on the western blot and no C3a or C5a were detected in the supernatant. Upon exposure to normal human serum, siFH cells deposited more complement activation fragments, compared to siC (data not shown). Therefore, FH, produced by the A549 cells was functionally active and controlled complement. Since the deposits of ext-FH were found only in few patients, survival analysis was not possible. Therefore, we set up a mouse model, in which we inoculated lung cancer cell line TC1 into WT and hepatoFH−/−mice³¹. These mice have only 20% of FH produced by extrahepatic sources, which remained in the circulation (data not shown). TC-1 expressed very low levels of FH (data not shown). When TC-1 cells were inoculated in hepatoFH−/−mice, the level of FH inside the tumor was largely decreased (FIG. 3B), meaning that plasma FH is an important source of FH for the tumor. However, tumor growth in hepatoFH−/−mice was not modified compared to that in WT mice (data not shown). FH has been suggested as a neoangiogenesis inhibitor³². Nevertheless, no modification in vascular density or architecture or angiogenic gene expression was observed in tumors from hepatoFH−/−mice compared to those from WT mice (data not shown). Thus, as for ccRCC, the protumoral effect of FH on lung tumor cell phenotype is driven by a complement cascade independent mode of action.

Int-FH is Associated with a Poor Prognosis Value for Patients with Lung ADK

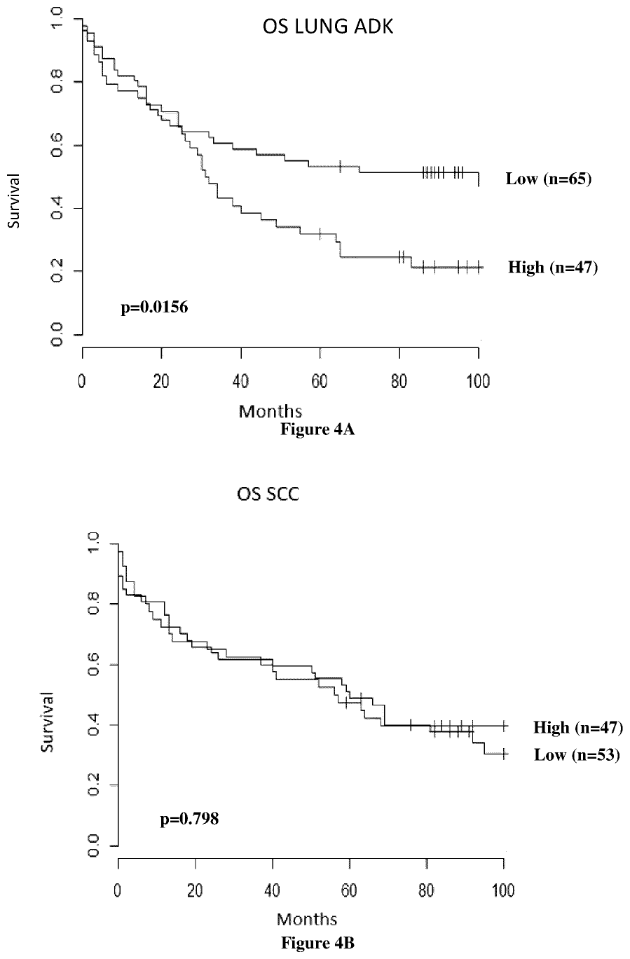

In NSCLC, we also detected int-FH. The pattern of int-FH staining was the same as that for ccRCC. NSCLC patients with either lung ADK or SCC from cohort 3, were classified into two groups according to the number of int-FH-positive cells (data not shown). In lung ADK, int-FH-positive cells were associated with a negative impact on OS (p=0.0156) (FIG. 4A). In contrast, in lung SCC, int-FH had no impact on OS (p=0.798) (FIG. 4B). The negative impact on lung ADK was independent of clinical or histological features and did not correlate with the immune infiltrate.

### DISCUSSION

Complement components form a plasma innate immune cascade but could also serve as multitasking proteins, as they have functions beyond this system. Here, we show that complement FH is locally expressed by multiple types of human tumors. We provide a paradigm shift for the impact of FH on cancer progression, showing a previously unrecognized, likely tumor cell-specific, intracellular function of FH outside the complement cascade, while the canonical, complement-regulatory function had no effect. Int-FH served as a driver of the proliferation and migration of ccRCC and lung ADK cells but not of normal cells or lung SCC cells. The presence of int-FH staining in tumor cells indicated poor prognosis for ccRCC and lung ADK.

It is important to emphasize that FH expression can be pro- or antitumoral according to the cancer type. In hepatocellular carcinoma, high expression of CFH is associated with a lower risk of recurrence³³, and high CFH is associated with improved survival, whereas CFH mutations are associated with worse survival¹⁸. This may be explained by the fact that FH is mainly produced by the liver and that low FH-expressing tumors will be the ones with the most dedifferentiated cells. Moreover, in addition to the cancer types, our data indicate that the histological subtype is also important to take into account. Indeed, while FH exerts a protumoral effect on lung ADK, no effects of FH were detected in the case of squamous cell lung cancer neither in patients nor in cell lines upon CFH silencing. Moreover, the effect will be context-dependent. Patients with int-FH positive lung ADK and ccRCC may benefit from int-FH targeting.

In conclusion, we show that int-FH exerts previously unrecognized functions, including modulation of the transcriptional activity of renal ccRCC and lung ADK cancer cells and effects on their proliferation, cell cycle, morphology and motility. Through these noncanonical functions, rather than through control of complement activation, int-FH confers poor prognosis in these types of cancer.

### REFERENCES

Throughout this application, various references describe the state of the art to which this invention pertains. The disclosures of these references are hereby incorporated by reference into the present disclosure.
1. Merle, N. S., Church, S. E., Fremeaux-Bacchi, V. & Roumenina, L. T. Complement System Part I—Molecular Mechanisms of Activation and Regulation. Front Immunol 6, 262 (2015).
2. Merle, N. S., Noe, R., Halbwachs-Mecarelli, L., Fremeaux-Bacchi, V. & Roumenina, L. T. Complement System Part II. Role in Immunity. Front. Immunol. 6, (2015).
3. Ricklin, D., Mastellos, D. C., Reis, E. S. & Lambris, J. D. The renaissance of complement therapeutics. Nat Rev Nephrol 14, 26-47 (2018).
4. Roumenina, L., Daugan, M. V., Petitprez, F., Sautes-Fridman, C. & Fridman, W. H. Context-dependent roles of complement in cancer. Nature Reviews Cancer (2019).
5. Lubbers, R., van Essen, M. F., van Kooten, C. & Trouw, L. A. Production of complement components by cells of the immune system. Clin. Exp. Immunol. 188, 183-194 (2017).
6. Bulla, R. et al. C1q acts in the tumour microenvironment as a cancer-promoting factor independently of complement activation. Nature Communications 7, 10346 (2016).
7. Roumenina, L. T. et al. Tumor Cells Hijack Macrophage-Produced Complement Clq to Promote Tumor Growth. Cancer Immunol Res (2019) doi:10.1158/2326-6066.CIR-18-0891.
8. Al-Adnani, M. S. & McGEE, J. O. Clq production and secretion by fibroblasts. Nature 263, 145-146 (1976).
9. Kesselring, R. et al. The complement receptors CD46, CD55 and CD59 are regulated by the tumour microenvironment of head and neck cancer to facilitate escape of complement attack. Eur. J. Cancer 50, 2152-2161 (2014).
10. Ajona, D. et al. Expression of complement factor H by lung cancer cells: effects on the activation of the alternative pathway of complement. Cancer Res. 64, 6310-6318 (2004).
11. Fishelson, Z. & Kirschfink, M. Complement C5b-9 and Cancer: Mechanisms of Cell Damage, Cancer Counteractions, and Approaches for Intervention. Front Immunol 10, 752 (2019).
12. Markiewski, M. M. et al. Modulation of the antitumor immune response by complement. Nat. Immunol. 9, 1225-1235 (2008).
13. Corrales, L. et al. Anaphylatoxin C5a creates a favorable microenvironment for lung cancer progression. J. Immunol. 189, 4674-4683 (2012).
14. Fan, Z., Qin, J., Wang, D. & Geng, S. Complement C3a promotes proliferation, migration and stemness in cutaneous squamous cell carcinoma. Journal of Cellular and Molecular Medicine 0, (2019).
15. Cho, M. S. et al. Autocrine effects of tumor-derived complement. Cell Rep 6, 1085-1095 (2014).
16. Bonavita, E. et al. PTX3 is an extrinsic oncosuppressor regulating complement-dependent inflammation in cancer. Cell 160, 700-714 (2015).
17. Bushey, R. T. et al. A Therapeutic Antibody for Cancer, Derived from Single Human B Cells. Cell Rep 15, 1505-1513 (2016).
18. Laskowski, J. et al. Complement factor H-deficient mice develop spontaneous hepatic tumors. J Clin Invest 130, (2020).
19. Arbore, G., Kemper, C. & Kolev, M. Intracellular complement—the complosome in immune cell regulation. Mol. Immunol. 89, 2-9 (2017).
20. Liszewski, M. K. et al. Intracellular Complement Activation Sustains T Cell Homeostasis and Mediates Effector Differentiation. Immunity 39, 1143-1157 (2013).
21. West, E. E., Kolev, M. & Kemper, C. Complement and the Regulation of T Cell Responses. Annual Review of Immunology 36, 309-338 (2018).
22. King, B. C. et al. Complement Component C3 Is Highly Expressed in Human Pancreatic Islets and Prevents β Cell Death via ATG16L1 Interaction and Autophagy Regulation. Cell Metab. 29, 202-210.e6 (2019).
23. Junnikkala, S. et al. Secretion of soluble complement inhibitors factor H and factor H-like protein (FHL-1) by ovarian tumour cells. British Journal of Cancer 87, 1119 (2002).
24. Gasque, P. et al. Expression of complement components of the alternative pathway by glioma cell lines. J. Immunol. 149, 1381-1387 (1992).
25. Junnikkala, S. et al. Exceptional resistance of human H2 glioblastoma cells to complement-mediated killing by expression and utilization of factor H and factor H-like protein 1. J. Immunol. 164, 6075-6081 (2000).
26. Riihilä, P. M. et al. Complement factor H: a biomarker for progression of cutaneous squamous cell carcinoma. J. Invest. Dermatol. 134, 498-506 (2014).
27. Smolag, K. I. et al. Complement inhibitor factor H expressed by breast cancer cells differentiates CD14+ human monocytes into immunosuppressive macrophages. Oncolmmunology 9, 1731135 (2020).
28. Józsi, M., Schneider, A. E., Kárpáti, É. & Sándor, N. Complement factor H family proteins in their non-canonical role as modulators of cellular functions. Seminars in Cell & Developmental Biology 85, 122-131 (2019).
29. Seol, H. S. et al. Complement proteins C7 and CFH control the stemness of liver cancer cells via LSF-1. Cancer Lett. 372, 24-35 (2016).
30. Ajona, D., Hsu, Y.-F., Corrales, L., Montuenga, L. M. & Pio, R. Down-Regulation of Human Complement Factor H Sensitizes Non-Small Cell Lung Cancer Cells to Complement Attack and Reduces In Vivo Tumor Growth. The Journal of Immunology 178, 5991-5998 (2007).
31. Vernon, K. A. et al. Partial Complement Factor H Deficiency Associates with C3 Glomerulopathy and Thrombotic Microangiopathy. J. Am. Soc. Nephrol. 27, 1334-1342 (2016).
32. Liu, J. & Hoh, J. Loss of Complement Factor H in Plasma Increases Endothelial Cell Migration. J Cancer 8, 2184-2190 (2017).
33. Yang, J. D. et al. Genes associated with recurrence of hepatocellular carcinoma: integrated analysis by gene expression and methylation profiling. J. Korean Med. Sci. 26, 1428-1438 (2011).
34. Martin, M et al. Factor H uptake regulates intracellular C3 activation during apoptosis and decreases the inflammatory potential of nucleosomes. Cell death and differentiation vol. 23,5 (2016): 903-11.
35. Vernon, K. A. et al. Partial Complement Factor H Deficiency Associates with C3 Glomerulopathy and Thrombotic Microangiopathy. J. Am. Soc. Nephrol. 27, 1334-1342 (2016).
36. Liu, J. & Hoh, J. Loss of Complement Factor H in Plasma Increases Endothelial Cell Migration. J Cancer 8, 2184-2190 (2017).
37. Yang, J. D. et al. Genes associated with recurrence of hepatocellular carcinoma: integrated analysis by gene expression and methylation profiling. J. Korean Med. Sci. 26, 1428-1438 (2011).

1. A method for predicting the survival time of a patient suffering from a cancer and treating the patient, comprising i) determining in a sample obtained from the patient the expression level of int-FH and ii) treating a patient identified as having an expression level higher than its predetermined reference value with an inhibitor of int-FH.

2. (Canceled)

3. The method according to claim 1, wherein the cancer is a lung adenocarcinoma or a renal carcinoma.

4. The method according to claim 3, wherein the renal carcinoma is a clear cell renal cell carcinoma.

5. The method according to claim 1, comprising i) determining in a sample obtained from the patient the expression level of int-FH ii) comparing the expression level determined at step i) with its predetermined reference value and iii) providing a good prognosis when the expression level determined at step i) is higher than its predetermined reference value, or providing a bad prognosis when the expression level determined at step i) is lower than its predetermined reference value.

6. The method according to claim 1, wherein the cancer is a liver cancer.

7. (canceled)

8. (canceled)

9. The method according to claim 1, wherein said inhibitor of int-FH is a siFH.

10. The method according to claim 1, wherein said inhibitor of int-FH is a single domain antibody directed against int-FH.

11. A pharmaceutical composition comprising an inhibitor of int-FH for use in the treatment of a cancer.

12. (canceled)

13. The pharmaceutical composition according to claim 11, containing at least one vehicle which is pharmaceutically acceptable to be injected directly into a tumor.

14. The pharmaceutical composition according to claim 13, wherein said at least one vehicle is a liposome.

15. The pharmaceutical composition according to claim 13, wherein said at least one vehicle is a targeting liposome.

16. The pharmaceutical composition according to claim 13, wherein the tumor is a lung adenocarcinoma or a clear cell renal cell carcinoma.
